(19) United States
(12) Plant Patent Application Publication
(10) Pub. No.: US 2007/0079409 P1
(43) Pub. Date: Apr. 5, 2007

(54) Motivational message tree

(76) Inventor: Mark Tabron, Cheverly, MD (US)

Correspondence Address: RADER FISHMAN & GRAUER PLLC, LION BUILDING, 1233 20TH STREET N.W., SUITE 501, WASHINGTON, DC, 20036, US

(21) Appl. No.: 11/242,892

(22) Filed: Oct. 5, 2005

(51) Int. Cl. A01H 5/00

(52) U.S. Cl. PLT/221

(57) ABSTRACT

The apparatus of the present invention is directed to a motivational tree designed to inspire, educate, encourage, uplift, and/or motivate persons of any age. The motivational tree includes a foliage portion have a plurality of leaves, a trunk having a plurality of extending branches a first end to which the plurality of leaves are attached and a soil area on a second end, and a pot portion that is securely connected to the soil area of said trunk, wherein the pot portion includes a playback circuit that outputs an audible motivational message when activated by one of the plurality of leaves. The playback circuit includes a communication port that provides the capability to download motivational message data from an external processor that may be connected to a network. Further, the playback circuit has a memory device that includes internal memory and a removable memory.

### BACKGROUND OF THE INVENTION

The present invention is related to a motivational device and in particular to a motivational message tree that outputs a variety of motivational and inspirational messages that are designed to inspire, educate, motivate, uplift, and/or encourage a user.

The public is bombarded with negative messages on a daily basis. These messages, some of which encourage destructive behavior, are provided through television and radio programming, and music. Tools or devices that provide an encouraging or motivational message can make a tremendous difference in minimizing the effects of negative messages in the lives of persons both young and old.

A wide variety of toys, dolls, and figurines have been developed for this specific purpose. Many of these products, however, are primarily designed for the entertainment and amusement of kids. Moreover, because of the appearance of these products, most cannot be placed in conspicuous areas of the home or office.

Therefore, the need exists for a device that can provide soothing and uplifting messages to a person of any age, and at the same time be a natural fixture in the home or office setting.

### BRIEF SUMMARY OF THE INVENTION

To overcome the above problems and other disadvantages of the prior art, an object is configured in the form of a tree to provide motivational messages through a playback circuit for the purpose of inspiring, educating, uplifting, and encouraging a user.

In an embodiment of the present invention, a motivational tree comprises a foliage portion have a plurality of leaves; a trunk having a plurality of extending branches a first end to which the plurality of leaves are attached and a soil area on a second end; and a pot portion that is securely connected to the soil area of said trunk, wherein the pot portion includes a playback circuit that outputs the audible motivational message when activated by one of the plurality of leaves.

In another embodiment of the present invention, a faux plant comprises a foliage portion having a plurality of leaves, wherein an activation switch is located inside at least one of the plurality of leaves of the foliage portion; a base portion connected to the foliage portion through a plurality of branches and a trunk, wherein the base portion includes a playback circuit, and wherein a processor of the playback circuit is connected to the activation switch through wiring, and wherein the playback circuit outputs an audible message based on data selected by the processor when the activation switch is activated.

### BRIEF DESCRIPTION OF THE DRAWINGS

FIG. 1 is a diagram illustrating a motivational tree consistent with the present invention;

FIG. 2 is a diagram illustrating a cavity of a base portion of the motivational tree consistent with the present invention;

FIG. 3 is a diagram illustrating a bottom of a base portion of the motivational tree consistent with the present invention;

FIG. 4 is a block diagram illustrating a playback circuit consistent with the present invention; and

FIG. 5 is a block diagram illustrating a power supply consistent with the present invention; and

FIG. 6 is a block diagram illustrating memory consistent with the present invention.

### DETAILED DESCRIPTION OF THE INVENTION

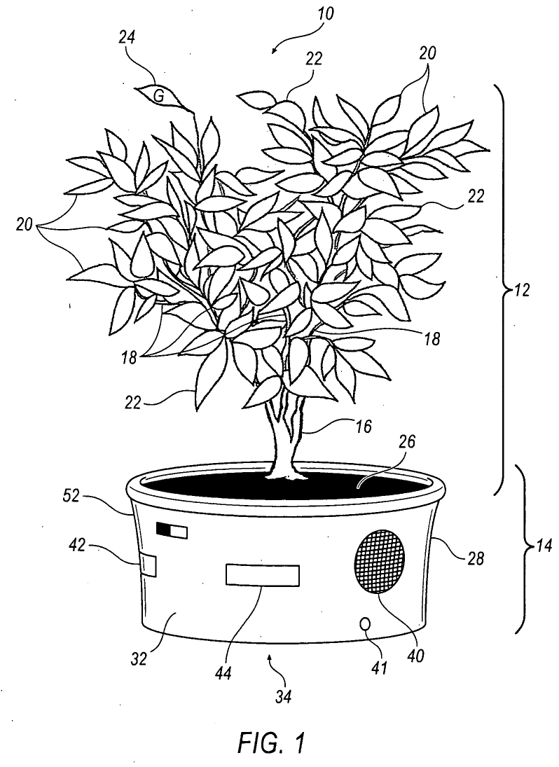

FIGS. 1-3 illustrate various views of a motivational tree 10 consistent with an embodiment of the present invention. The motivational tree 10 includes a foliage portion 12 and a base portion 14.

The foliage portion 12 has the appearance of a faux tree. This faux tree may be in the form and have the appearance of any one of the variety of trees that exist in nature, or of a tree produced through an artistic impression or interpretation. In this example, the foliage portion 12 is in the form of a bonsai tree. In the bonsai tree example, the foliage portion 12 includes a trunk 16, a plurality of branches 18, and a plurality of leaves 20, all of which are made of plastic or known synthetic materials normally used in the production of faux plant products. The trunk 16 and the plurality of branches are hollow and in addition to their aesthetic purpose, serve as a conduit for wiring (not shown) used in circuitry that will be described in detail below. The plurality of leaves 20 include dummy leaves 22 and at least one golden leaf 24. The dummy leaves 22 have only an aesthetic purpose. The golden leaves 24 include a mechanism that activates motivational message playback.

The base portion 14 is affixed to the bottom of the trunk 16. The base portion includes a soil area 26 and a pot or planter 28. The soil area 26 is formed as the top of the base portion 14 and surrounds the bottom of the trunk 16. The soil area 26 simulates for example, the topsoil that in which a natural plant, or bonsai plant would be planted.

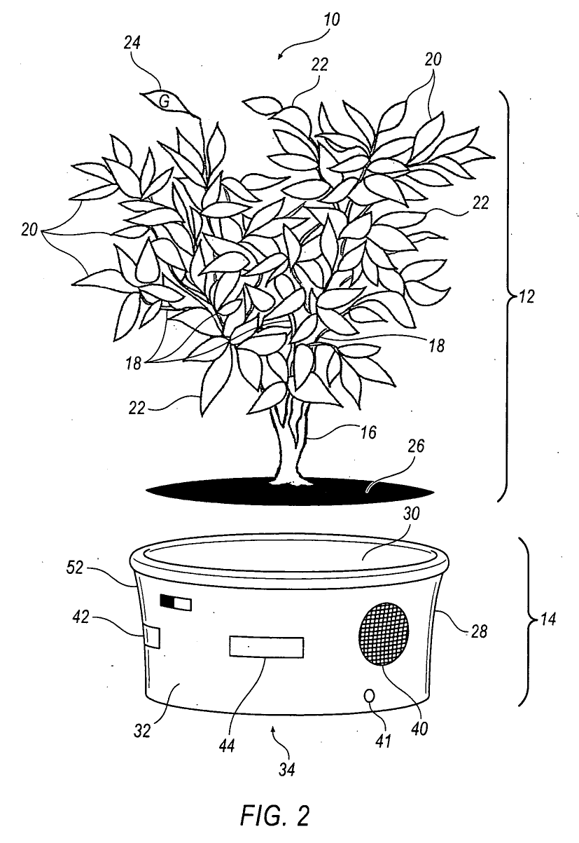

As shown in FIG. 2, the pot 28 can be in any of a variety of shapes and sizes and includes a cavity 30, a pot side 32, and a pot bottom 34. The pot 28 is fixedly attached to the soil area 26, such that the soil area 26 securely fits into the cavity 30 and serves as the top of the base portion 14. A playback circuit 36 of the motivational message tree 10 is housed within the cavity 30 and a pot side 32 of the pot 28. Various components of a playback circuit 36 facilitate user interaction and are integrated into the pot side 32 and the pot bottom 34.

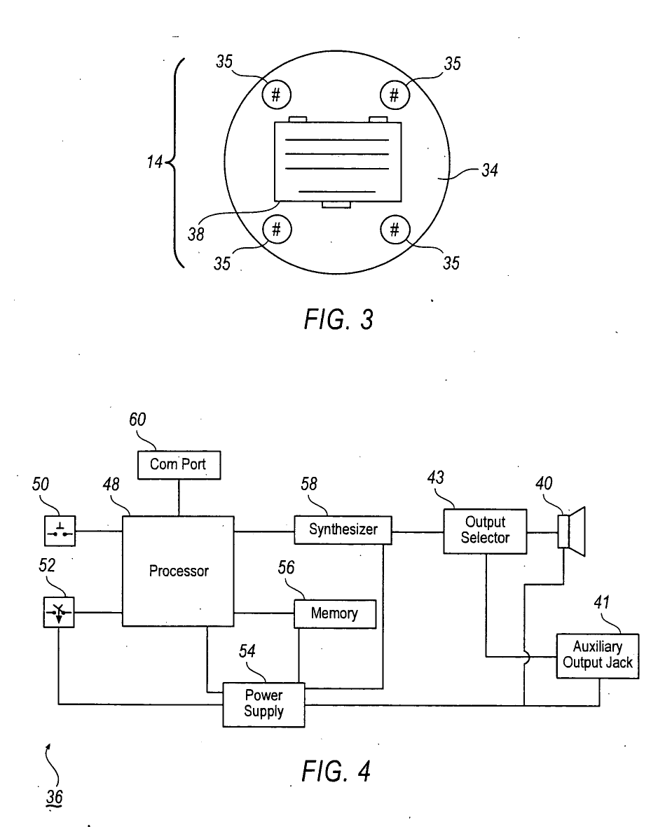

FIG. 3 illustrates a battery compartment 36 that is integrated into the pot bottom 34. The battery compartment 38 holds four D-cell batteries, for example, but may be modified as needed to hold any suitable size or type of battery device. The pot bottom 34 further includes a plurality padded portions 35 that prevent the motivational tree 10 from sliding on most surfaces. The plurality of padded portions 35 further raise the pot bottom 34 so that the surface on which the motivational tree 10 rests is not damaged.

FIGS. 1 and 2 further illustrate a speaker 40, a communication port 42, and a removable media compartment 44 integrated into the pot side 32. The speaker 40 provides audio output to the user. The communication port 42 provides connection capability to an external processor or device used for data transfer. The removable media compartment 44 provides a docking mechanism for an external memory device or data cartridge. The pot side 32 further includes a power switch 52 that enables the playback circuit 36 to be turned on or off. These components will be described in further detail below.

FIG. 4 is a block diagram illustrating the playback circuit 36 consistent with an embodiment of the present invention.

The playback circuit 36 enables the motivational tree 10 to output motivational messages to a user. The playback circuit 36 includes the speaker 40, an auxiliary output jack 41, the communication port 42, an output selector 43, a processor 48, an activation switch 50, the power switch 52, a power supply 54, a memory 56, and a synthesizer 58.

The processor 48 controls the playback of the motivational message. The processor 48 is connected to the memory 58 so that it may access data stored in the memory 58. The processor 48 uses the accessed data to perform operational functions or the processor 48 may output the accessed data to the synthesizer 60. In this manner, the processor 48 is configured to control the synthesizer 60 in the generation of speech based on data output from the memory 56.

The activation switch 50 is a spring-loaded depressible switch that is located within the golden leaf 24, however, another suitable switch type may be used to perform the same function. The activation switch 50 is connected to the processor 48, through wiring located within the hollow portion of the plurality of branches 18 and the trunk 16.

The power switch 52 activates the power supply 54 so that the required voltage and current may be provided to the speaker 40, processor 48, the memory 56, and the synthesizer 60. The power switch 52 is a slidable contact switch, however, another suitable switch type may be used to perform the same function.

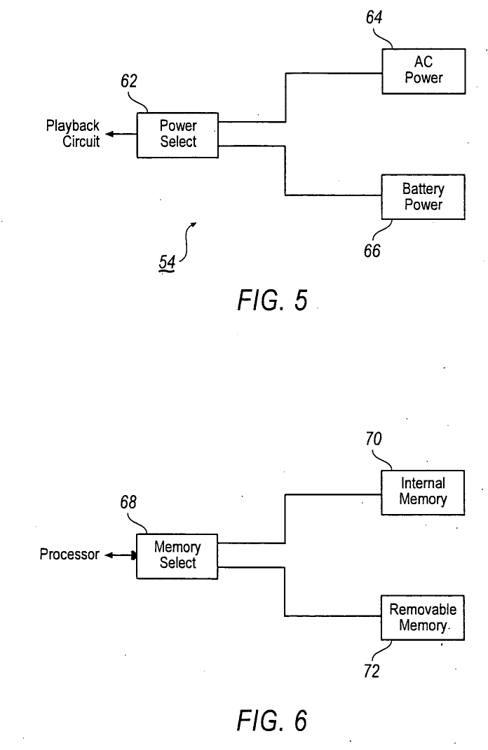

FIG. 5 is a block diagram illustrating the power supply 54 consistent with an embodiment of the present invention. The power supply 54 includes a power selector 62, an AC power source 64, and a battery power source 66. The AC power source 64 supplies power through a connection to an electrical outlet. The battery power source 66 supplies power through four D-cell batteries or other suitable battery source that are housed in the battery compartment 38. The power selector 62 supplies power to the processor 48 from either the AC power source 64 or the battery power source 66 based upon the power levels detected from each device. For example, when the power selector 62 detects power from the AC power source 64, then the power selector 62 selects the AC power source 64 to provide power to the speaker 40, the processor 48, the memory 56, and the synthesizer 60, even if the battery power source 64 is suitably charged. However, if power from the AC power source 64 is interrupted or falls below a predetermined level, then the power selector 62 will select the battery power source 64 to provide power to the speaker 40, processor 48, the memory 56, and the synthesizer 60.

FIG. 6 is a block diagram illustrating the memory 56 consistent with an embodiment of the present invention. The memory 56 stores digital voice data that is processed by the processor 48 and provided to the synthesizer 60 for output through the speaker 40. The memory 56 includes a memory selector 68, an internal memory 70, and a removable memory device 72. The internal memory 70 stores speech data and/or other data used to uplift, encourage, or motivate a user. Furthermore, the internal memory 70 stores an operating program of the processor 48 and any other executable data used by the processor 48 to operate the motivational message tree 10. The removable memory device 72 is an interchangeable cartridge that stores speech data and/or other data used to uplift, encourage, or motivate a user. The removable memory device 72 also stores the operating program of the processor 48 and any other executable data used by the processor 48 to operate the motivational message tree 10. The removable memory device 72 is manually inserted into and securely rests inside the removable media compartment 44. The memory selector 68 enables the processor 48 to access speech or operational data stored on the internal memory 70 or the removable memory device 72. For example, when the removable memory device 48 is not installed into the removable media compartment 44, then the memory selector 68 will enable the processor 48 to access the internal memory 70. Alternatively, when the removable media device 72 is installed in the removable media compartment 44, then the memory selector 68 will enable the processor 36 to access only the data stored on the removable memory device 72.

The synthesizer 58 is controlled by the processor 48. The synthesizer 58 generates analog speech signals based on the digital data stored in the memory 56. The synthesizer 58 outputs the speech signals to the speaker 40. Power is supplied to the synthesizer through the power supply 54.

The speaker 40 and the auxiliary output jack 41 are connected to receive an output from the synthesizer 58 through the output selector 43. Both the speaker 40 and an external audio output device (not shown) which is plugged into the auxiliary output jack 41 reproduce the speech data stored in the memory 56 based upon the analog speech signals received from the synthesizer 58. The output selector 43 is a switch that connects the speaker the output of the synthesizer 58 when the external audio output device is not plugged into the auxiliary output jack 41. When the external audio output device is plugged into the auxiliary output jack 41, the output selector 43 connects the auxiliary output jack 41 to the output of the synthesizer 58 so that the external audio output device reproduces the output of the synthesizer 58. The external audio output device may include, for example, a speaker, headphones, or the like.

The communication port 60 enables the motivational tree 10 to be connected to an external device. The communication port 60 allows speech data and other motivational data to be downloaded to the internal memory 70 from an external processor or computer. The communication port 60 uses the Universal Serial Bus (USB) external bus standard, which provides plug-and-play capabilities such that the processor 48 may communicate seamlessly with the external processor. The communication port enables data to be downloaded to the internal memory 70 from an external processor connected to a network such as the Internet.

Based on the configuration described above, the motivational message tree 10 outputs an audible motivational message through the speaker 40, when the golden leaf 24 is depressed. In particular, depressing the golden leaf 24 enables the processor 48 to randomly select a motivational message from digital data stored in the memory 56. The processor 48 outputs the selected speech data to the synthesizer 58. The synthesizer 58 generates an analog speech signal based on the digital speech data received from the processor 48. The generated speech signal is outputted to the speaker 40 so that the audible message is presented to the user. Further, motivational messages may be downloaded to the motivational message tree 10 through the communication port 60 from an external processor connected to a network such as the Internet.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A motivational tree that outputs an audible motivational messages, said tree comprising:
a foliage portion have a plurality of leaves;
a trunk having a plurality of extending branches a first end to which the plurality of leaves are attached and a soil area on a second end; and
a pot portion that is securely connected to the soil area of said trunk, wherein the pot portion includes a playback circuit that outputs an audible motivational message when activated by one of the plurality of leaves.

2. The motivational tree of claim 1, wherein said one of the plurality of leaves includes a depressible switch.

3. The motivational tree of claim 1, wherein said plurality of branches and said trunk are hollow and provide a conduit for wiring of said playback circuit.

4. The motivational tree of claim 1, wherein said playback circuit includes a processor, a memory, a synthesizer, and a speaker.

5. The motivational tree of claim 4, wherein said memory includes at least one of an internal memory and a removable memory device.

6. The motivational tree of claim 4, wherein said playback circuit further includes a communication port based on a USB standard.

7. The motivational tree of claim 4, wherein said memory stores speech data and operational data.

8. The motivational tree of claim 4, wherein said tree is a bonsai tree.

9. The motivational tree of claim 4, wherein the playback circuit includes a communication port that enables speech data to be downloaded to the memory from an external processor connected to a network.

10. The motivational tree of claim 1, wherein components of the playback circuit are integrated into a side and bottom of the pot portion.

11. A faux plant, comprising:
a foliage portion having a plurality of leaves, wherein an activation switch is located inside at least one of the plurality of leaves of the foliage portion;
a base portion connected to the foliage portion through a plurality of branches and a trunk, wherein the base portion includes a playback circuit, and
wherein a processor of the playback circuit is connected to the activation switch through wiring, and
wherein the playback circuit outputs an audible message based on data selected by the processor when the activation switch is activated.
